(19) United States
(12) Patent Application Publication
(10) Pub. No.: US 2024/0016748 A1
(43) Pub. Date: Jan. 18, 2024

(54) DIRECT DELIVERY OF VITAMINS TO INHIBIT MICROBIAL PATHOGENS

(71) Applicant: DSM IP ASSETS B.V., Heerlen (NL)

(72) Inventors: Thanh-Van PHAM, Kaiseraugst (CH); Ateequr REHMAN, Kaiseraugst (CH); Robert STEINERT, Kaiseraugst (CH); Wilbert SYBESMA, Kaiseraugst (CH)

(21) Appl. No.: 18/250,377

(22) PCT Filed: Oct. 26, 2021
(86) PCT No.: PCT/EP2021/079717

§ 371 Date: Apr. 25, 2023

(30) Foreign Application Priority Data
Oct. 28, 2020 (EP) 20204234.7

(51) Int. Cl. A61K 9/28; A61K 31/375; A61K 31/4415; A61K 31/525; A61K 31/455; A61K 31/197; A61P 31/04; A61K 9/48

(52) U.S. Cl. CPC A61K 9/2866; A61K 31/375; A61K 31/4415; A61K 31/525; A61K 31/455; A61K 31/197; A61P 31/04; A61K 9/4891

(57) ABSTRACT

This invention relates to the direct delivery of a vitamin composition to the large intestine, in order to decrease the growth of pathogenic organisms in the gut, such as Adherent Invasive E. coli (AIEC), Salmonella enteriditis and Clostridium difficile. The vitamin composition comprises a combination of the following vitamins: Vitamin C, Vitamin B2, Vitamin B3, Vitamin B5, Vitamin B6 and folic acid.

### BRIEF DESCRIPTION OF THE INVENTION

This invention relates to the direct delivery of vitamins to the intestine to discourage the growth of pathogenic organisms such as Adherent Invasive E. coli (AIEC), Clostridium difficile, including toxigenic and non-toxigenic C. difficile, and Salmonella enteritidis. This method can be used to treat or prevent microbial diseases, such as food poisoning and traveler's diarrhea, as well as discouraging pathogenic growth after the use of antibiotics. The vitamin composition may comprise Vitamin C as a sole active agent; Vitamin C in combination with Vitamin B2 and/or B3; or Vitamin C in combination with Vitamin B2, Vitamin B3, Vitamin B5, Vitamin B6 and folic acid.

### BACKGROUND OF THE INVENTION

Direct delivery of various vitamins and other active ingredients to the gut has been described. See, e.g. U.S. Pat. No. 9,433,583 B2 directed to a colon-targeted single dosage form comprising vitamin D and optionally further vitamins for preventing colorectal adenomatous polyps and colorectal cancer and WO2014/070014 directed to the use of riboflavin (Vitamin B2) to stimulate the population of Faecalibacterium prausnitzii.

The use of broad spectrum antibiotics, such as penicillins/cephalosporine, fluoroquinolines, and clindamycin will alter the populations of the gut microbiome bacteria. When the antibiotic kills off the unwanted bacteria, the remaining population will have less competition for space and nutrients. The net effect can result in a more extensive growth than normal of certain pathogenic bacteria which occasionally reside in the gut, albeit at a low level. Examples of such bacteria include Adherent Invasive Escherichia coli “AIEC”, (estimated to be present in approximately 20% of humans) and Clostridium difficile (estimated to be present in approximately 3% of humans). Other factors which can favor the increase in endogenous pathogens include stress, changing diet, and aging.

More commonly, a source of bacterial pathogens is ingestion or poor hygienic measures. An example is food poisoning, which can be caused by a number of bacteria including Campylobacter spp, Clostridium perfringens, various strains of E. coli, Listeria spp., Salmonella spp., Bacillus cereus, Shigella spp, Staphylococcus aureus, and Vibrio spp. Symptoms include nausea, diarrhea, vomiting, stomach cramping, a high temperature (380 or above), and feeling generally unwell (feeling tires, having aches or chills).

Another consequence of bacterial pathogens is so-called “traveler's diarrhea” which can affect up to 20% of those who travel to high risk destinations. Bacteria which are often the culprit are E. coli, Campylobacter jejuni, Shigella spp, and Salmonella spp. Symptoms include diarrhea, often accompanied by at least one of: fever, nausea, vomiting, cramps or bloody stools. Approximately 13% of travelers are confined to bed for 1-3 days, and some 12-46 percent have to change their itinerary. In a small percentage hospitalization is required.

AIEC is associated with the pathogenesis of a number of diseases, including inflammatory bowel disease (Crohn's disease and ulcerative colitis). Clostridium difficile infection causes a number of conditions, ranging from mild diarrhea to more severe and even life-threatening conditions such as pseudomembranous colitis, toxic megacolon, perforation of the colon, and sepsis.

Salmonella infections can produce symptoms such as vomiting, fever and stomach cramps. These can vary in severity and can even lead to hospitalization and even death. One source of Salmonella infection is ingestion of tainted food.

It would be desirable to have an all-natural, easy to use composition which can inhibit the growth of pathogenic bacteria in the intestines.

### DETAILED DESCRIPTION OF THE INVENTION

It has been found, in accordance with this invention that a composition comprising Vitamin C as the sole active ingredient when administered directly to the gut, will inhibit the population of pathogenic bacteria. The Vitamin C may also be combined with other active ingredients, such as Vitamin B2 and Vitamin B3. Also, a combination of Vitamin C, Vitamin B2, Vitamin B3, Vitamin B5, Vitamin B6 and folic acid (herein after referred to as “Vitamin Mix”) is also effective.

While not wishing to be bound by theory, it is believed that the Vitamin C may work by multiple means to inhibit pathogens. First, it may enhance the production of Short Chain Fatty Acids (SCFAs), thus contributing to the overall anti-pathogenic effect observed. Secondly the Vitamin C, which has antioxidant properties, may decrease the local availability of oxygen to pathogens, thus limiting their growth. Further, Vitamin C may also lower the physiological pH in the intestine, which may contribute to the growth inhibitory effect.

Thus, one embodiment of this invention is a method of decreasing a population of pathogenic bacteria present in the intestine comprising administering a composition comprising Vitamin C directly to the intestine. Another embodiment of this invention is a method of decreasing a population of pathogenic bacteria present in the intestine comprising administering a composition comprising Vitamin C, Vitamin B2 and Vitamin B3 directly to the intestine. Another embodiment of this invention is a method of decreasing a population of pathogenic bacteria present in the intestine comprising administering a composition comprising of Vitamin C, Vitamin B2, Vitamin B3, Vitamin B5, Vitamin B6 and folic acid (“Vitamin Mix”) directly to the large intestine.

One embodiment of this invention is the use of a directly delivered composition comprising Vitamin C to decrease the population of pathogenic bacteria in the distal gut. In some embodiments the composition used comprises Vitamin C, Vitamin B2 and Vitamin B3. In some embodiments, a Vitamin Mix is used to decrease the population of pathogenic bacteria in the distal gut. Another embodiment is the use of directly delivered Vitamin Mix to prevent or treat diseases or adverse conditions caused by pathogenic bacteria in the distal gut. Another embodiment is the use of directly delivered Vitamin Mix to prevent or treat traveler's diarrhea, food poisoning and antibiotic associated diarrhea (AAD).

As AIEC is involved in the development of certain inflammatory bowel diseases such as Crohn's disease and ulcerative colitis, Salmonella is associated with diarrhea and food poisonings, and Clostridium difficile infection is involved in a number of conditions ranging from mild diarrhea to more severe and even life threatening conditions such as pseudomembranous colitis, toxic megacolon, perforation of the colon, and sepsis, the direct delivery of the aforesaid combination of vitamin(s) can prevent or lessen the risk of development of these adverse conditions and diseases in an individual who is at risk of developing any of these adverse conditions and diseases. In some embodiments the person at risk is one who is at risk of developing traveler's diarrhea, food poisoning, or is exposed to antibiotics.

Thus, another embodiment is a method of preventing, reducing the risk or delaying the onset of a disease or adverse condition; methods of treating a disease or adverse condition; and methods of meeting the nutritional needs of a person experiencing a disease or adverse condition, wherein the disease or adverse condition is associated with a pathogenic bacteria in the intestine. In some embodiments, the disease or adverse condition is selected from the group consisting of: inflammatory bowel disease (Crohn's disease and ulcerative colitis), Clostridium difficile-related diarrhea, pseudomembranous colitis, toxic megacolon, perforation of the colon, sepsis, bacterial food poisoning, and bacterial-related traveler's diarrhea and AAD comprising directly delivering to a person exposed to an antibiotic, a composition comprising an active ingredient selected from the group Vitamin C; the combination of Vitamin C, Vitamin B2 and Vitamin B3; and the combination of Vitamin B2, Vitamin B3, Vitamin B5, Vitamin B6 and folic acid “Vitamin Mix”; characterized in that the composition is directly delivered to the large intestine.

### BRIEF DESCRIPTION OF THE FIGURES

FIG. 1 shows the experimental setup. FIG. 1A shows the experimental setup of the in vitro fermentation experiment. FIG. 1B shows the experimental setup of the pathogen challenge test.

FIG. 2. Effect of the combination of vitamins (“Vitamin Mix”) on pathogen levels. FIG. 2A shows the levels of A/EC upon administration of Vitamin Mix, when dosed to the healthy (without exposure to antibiotic) and dysbiosed microbiota (previously treated with antibiotics). FIG. 2B shows the levels of Salmonella enteritidis upon administration of Vitamin Mix, when dosed to the healthy (without exposure to antibiotic) and dysbiosed microbiota (previously treated with antibiotics). FIG. 2C shows the levels of Clostridium difficile upon administration of Vitamin Mix compared to the counterpart control, when dosed to the healthy (without exposure to antibiotic) and dysbiosed microbiota (previously treated with antibiotics).

### DEFINITIONS: AS USED THROUGHOUT, THE FOLLOWING DEFINITIONS APPLY

The term “Vitamin Mix” means the combination of Vitamin C, Vitamin B2, Vitamin B3, Vitamin B5, Vitamin B6 and Folic Acid.

The term “vitamin B2” which can be used interchangeably with “riboflavin”, includes riboflavin and esters thereof, in particular riboflavin-5′-phosphate and other pharmaceutically acceptable forms.

The term “vitamin C” which can be used interchangeably with “ascorbic acid” also includes pharmaceutically acceptable salts thereof (e.g. sodium ascorbate and calcium ascorbate) and pharmaceutically acceptable esters thereof (in particular ascorbyl palmitate) and other pharmaceutically acceptable forms.

The term “Vitamin B3” which can be used interchangeably with “niacinamide” and “niacin” also includes nicotinic acid and other pharmaceutically acceptable forms. The term “Vitamin B5” which can be used interchangeably with “pantothenic acid” also includes coenzyme A, phosphopantetheine, pantetheine and other pharmaceutically acceptable forms.

The term “Vitamin B6” which can be used interchangeably with “pyridoxine” also includes pyridoxine phosphate, pyridoxal, pyridoxal phosphate and other pharmaceutically acceptable forms.

The term “Folic Acid” which can be used interchangeably with “Vitamin B9” also includes folate, 5-methyl-tetrahydrofolate, monoglutamate folate, polyglutamate folate and other pharmaceutically acceptable forms.

“Decreasing the population” of pathogenic bacteria means that the amount of pathogenic bacteria present in a gut which has been exposed to a Vitamin or Vitamin combination of this invention is decreased compared to a microbiome which has not been exposed to the Vitamin or Vitamin combination of this invention.

“Pathogenic microorganisms” means at least one species selected from the group consisting of: AIEC, Clostridium difficile, Salmonella spp., Campylobacter spp., Clostridium perfringens, E. coli, Listeria spp., Bacillus cereus, Shigella spp, Staphylococcus aureus, Enterococcus spp., Streptococcus spp., Klebsiella spp., and Vibrio spp.

“Direct delivery” or “directly delivered” means that the vitamin or combination of vitamins is administered in a manner such that the vitamin(s) is not absorbed in the stomach and/or small intestine; rather the vitamin(s) is present in the distal intestinal tract, preferably the large intestine, where it is available to the microbiome. The vitamin(s) not part of a person's usual daily nutritional requirements (generally obtained through diet and conventional vitamin supplementation), and are administered in excess thereof. For human use, the preferred method is through a form which delays release until the large intestinal tract is reached. Alternatively included is a method of administering a large enough dose so that only a portion of the vitamin delivered is absorbed in the stomach, and the remainder which is an effective dose, is available to the large intestinal tract; although not preferred, this method of delivery can be used for humans as well.

“Prevent” can include lessening the risk of an adverse condition occurring, lessening the symptoms of an adverse condition, lessening the severity of an adverse condition and prolonging the time for occurrence of an adverse condition.

Exposure to Antibiotics

We have found that Vitamin C, the combination of Vitamin C, Vitamin B2 and B3, and the Vitamin Mix can decrease the abundance of pathogenic bacteria after the gut microbiome has been exposed to antibiotics.

Thus, one embodiment of this invention is a method of decreasing the population of a pathogenic bacteria in a gut which is exposed to antibiotics comprising administering an active ingredient directly to the large intestine and the active ingredient is selected from the group consisting of Vitamin C, the combination of Vitamin C, Vitamin B2 and B3, and the Vitamin Mix. In some embodiments, the bacterial populations which are decreased are AIEC, Salmonella enteritidis and/or Clostridium difficile Another embodiment of this invention is the use of a directly delivered composition comprising an active ingredient selected from the group consisting of Vitamin C, the combination of Vitamin C, Vitamin B2 and B3, and Vitamin Mix, for the use in decreasing the population of a pathogenic bacteria in the gut of a person who has been exposed to an antibiotic. Preferably, the pathogenic bacteria is AIEC, Salmonella enteritidis and/or Clostridium difficile. This also includes the non-therapeutic use, and the prophylactic uses. Another embodiment of this invention is the use of a composition comprising an active ingredient selected from the group consisting of Vitamin C, the combination of Vitamin C, Vitamin B2 and B3, and Vitamin Mix, formulated for direct delivery to the gut microbiome of an animal in the large intestine, preferably a human, said animal having been exposed to an antibiotic, and characterized in that upon delivery to the large intestine, it results in a decreased population of AIEC, Salmonella enteritidis and/or Clostridium difficile in the gut microbiome.

Another embodiment of this invention is the use of a composition comprising an active ingredient selected from the group consisting of Vitamin C, the combination of Vitamin C, Vitamin B2 and B3, and Vitamin Mix in the manufacture of a medicament, nutraceutical or medical food for the use in decreasing the population of a pathogenic bacteria in the gut of a person who has been exposed to an antibiotic.

Another embodiment of this invention is an oral delivery formulation comprising a population decreasing-effective amount of a composition comprising an active ingredient selected from the group consisting of Vitamin C, the combination of Vitamin C, Vitamin B2 and B3, and Vitamin Mix, and said formulation is characterized in that the vitamin is delivered directly to the microbiome present in the large intestine.

Another embodiment of this invention is a medical food for persons who have received an antibiotic and who have a disease which can benefit from a decrease of a pathogenic bacteria in their gut.

Thus another aspect of this invention is a vitamin combination formulation comprising an active ingredient selected from the group consisting of Vitamin C, the combination of Vitamin C, Vitamin B2 and B3, and Vitamin Mix, and excipients; and said formulation is characterized in that the active ingredient is delivered directly to the gut microbiome in the large intestine, and is used for addressing the nutritional needs of a patient experiencing a disease/adverse condition characterized by an increased population of AIEC, Salmonella enteritidis or Clostridium difficile population in the gut microbiome following antibiotic exposure.

The antibiotic may be any antibiotic, including those which are known as broad spectrum antibiotics. Examples include, but are not limited to: penicillins/cephalosporine, fluoroquinolines, and clindamycin.

As antibiotics are known to affect the makeup of the gut microbiome for a prolonged period of time, the vitamin(s) composition may be administered up to two weeks prior to the administration of an antibiotic, concurrently with the administration of an antibiotic; or within one week, two weeks, within 4 weeks, or up to 90 days after administration of the antibiotic. A person “exposed to antibiotics” is someone who is currently receiving antibiotic treatment, who will be receiving antibiotic treatment within 2 weeks of receiving direct delivery of the vitamin(s) of this invention, or has already received antibiotic treatment within one week, two weeks, within 4 weeks, or up to 90 days after administration of the vitamin(s) of this invention.

Traveler's Diarrhea

Traveler's diarrhea is a general term for a common diarrheal illness often experienced by up to 60% of travelers during the first week or two of a stay in a foreign environment. It may be caused by a number of bacterial species, including E. coli (the most common), Campylobacter jejuni, Salmonella, and Shigella found in contaminated food/water in the new environment. It is characterized by an increase in frequency of bowel movements to three or more loose stools per day. We have found, in accordance with this invention, that Vitamin C, the combination of Vitamin C, Vitamin B2 and Vitamin B3, and Vitamin Mix can decrease the population of pathogenic organisms, including those which cause traveler's diarrhea.

Thus, one embodiment of this invention is a method of lessening the risk of, lessening the severity of, or treating traveler's diarrhea by directly administering an active ingredient selected from the group consisting of: Vitamin C, the combination of Vitamin C, Vitamin B2 and Vitamin B3, and Vitamin Mix to a person who is travelling, a person who will be travelling within one month, preferably within 2 weeks, or to a person who has travelled within the past two weeks.

Another embodiment of this invention is the use of an active ingredient selected from the group consisting of: Vitamin C, the combination of Vitamin C, Vitamin B2 and Vitamin B3, and Vitamin Mix to prevent or treat the symptoms of traveler's diarrhea, characterized in that the active ingredient is formulated so that it is directly delivered to the intestine. Another embodiment is the use of an active ingredient selected from the group consisting of: Vitamin C, the combination of Vitamin C, Vitamin B2 and Vitamin B3, and Vitamin Mix in the manufacture of a nutraceutical or pharmaceutical for the treatment or prevention of traveler's diarrhea, wherein the nutraceutical or pharmaceutical is directly delivered to the intestine. Another embodiment is a nutraceutical, pharmaceutical, or medical food to treat or lessen the symptoms of traveler's diarrhea, or to address the medical nutritional needs of a person at risk of, or suffering from traveler's diarrhea, comprising an active ingredient selected from the group consisting of: Vitamin C, the combination of Vitamin C, Vitamin B2 and Vitamin B3, and Vitamin Mix which is formulated so that it is directly delivered to the intestine.

Food Poisoning

Food poisoning symptoms include nausea, diarrhea, vomiting, stomach cramps, a high temperature (≥38° C. or above), and generally feeling unwell—such as having aches and chills and tiredness. These generally appear within a few hours of eating the offending food, but may not appear for a few weeks. Foods which may carry bacteria associated with food poisoning may not have been cooked or reheated thoroughly, not stored correctly (i.e. not been frozen or chilled), left at room temperature for too long, handled by someone who is ill, but did not sanitize properly, or eaten after its “use by” date.

The most common organisms associated with food poisoning include: Campylobacter, Salmonella and E. coli. Thus, one embodiment of this invention is a method of lessening the risk of, lessening the severity of, or treating food poisoning symptoms by directly administering an active ingredient selected from the group consisting of: Vitamin C, the combination of Vitamin C, Vitamin B2 and Vitamin B3, and the Vitamin Mix to a person who is at risk of eating a food contaminated with bacteria which cause food poisoning, or who has eaten such food.

Another embodiment of this invention is the use of an active ingredient selected from the group consisting of: Vitamin C, the combination of Vitamin C, Vitamin B2 and Vitamin B3, and Vitamin Mix to prevent or treat the symptoms of food poisoning comprising administering the active ingredient directly to the intestine of a person in need thereof. Another embodiment is the use of an active ingredient selected from the group consisting of: Vitamin C, the combination of Vitamin C, Vitamin B2 and Vitamin B3, and Vitamin Mix in the manufacture of a nutraceutical or pharmaceutical for the treatment or prevention of food poisoning, characterized in that the active ingredient is formulated so that it is delivered directly to the intestine.

Another embodiment is a nutraceutical, pharmaceutical, or medical food to treat or lessen the symptoms of food poisoning, or to address the medical nutritional needs of a person at risk of, or suffering from food poisoning comprising an active ingredient selected from the group consisting of: Vitamin C, the combination of Vitamin C, Vitamin B2 and Vitamin B3 and Vitamin Mix; and formulated so that the active ingredient is delivered directly to the intestine.

Animals

“Animals” include mammals, poultry and preferably humans. Preferred non-human animals are companion animals, and include dogs, cats, and horses. Among agriculturally important animals, preferred animals include poultry, swine, bovines, ovines and caprines and equines.

Additional Active Ingredients

The vitamin(s) of this invention may be used as the sole active ingredients, or may be combined with other active ingredients, such as conventional drug therapies, prebiotics, probiotics, and the like.

Doses:

The dosages used herein are intended to be in addition to the active ingredients that is ingested for general nutrition purposes. Instead, they act upon the gut microbiome environment as a whole, at the genus, species and strain level of the gut microbes. The active agents are not intended primarily to be metabolized directly by the animal, including the human rather they are intended primarily to be utilized by the bacterial population of the colon. Therefore, the amounts reported below would be consumed by the animal in addition to the usual diet, but as they are not directly available to the animal due to their delayed release.

Preferably, Vitamin B2 can be administered in an amount such that its local concentration in the colon is at least 0.01 g/L, preferably at least 0.1 g/L more preferably at 0.125 g/L. Preferred local concentrations in the colon range from about 0.1 g/L to about 0.5 g/L or from about 0.1 g/L to about 0.2 g/L, preferably about 0.125 g/L. Specific dosages per day can range up to 200 mg/day, preferably 5-100 mg/day, more preferably from 10-50 mg/day.

Preferably, Vitamin C can be administered in an amount such that its local concentration in the colon is at least 0.05 g/L, preferably at least 0.1 g/L, most preferably at least 2 g/L. Preferred local concentrations in the colon range from about 0.05 g/L to about 1.5 g/L, more preferably from about 0.5 g/L to about 1 g/L, most preferably from about 0.8 g/L to about 0.9 g/L. Specific dosages per day can range up to 2000 mg/day, preferably 100-2000 mg/day; more preferably 200-1000 mg/day.

Preferably, vitamin B5 can be administered in an amount such that its local concentration in the colon is at least 0.01 g/L, preferably at least 0.02 g/L, most preferably at least 0.04 g/L. Preferred local concentrations in the colon range from about 0.005 g/L to about 0.04 g/L, more preferably from about 0.015 g/L to about 0.025 g/L. Specific dosages per day can range up to up to 50 mg/day, preferably 1-50 mg/day; more preferably 5-25 mg/day.

Preferably, vitamin B3 can be administered in an amount such that its local concentration in the colon is at least 0.01 g/L, preferably at least 0.02 g/L, most preferably at least 0.04 g/L. Preferred local concentrations in the colon range from about 0.005 g/L to about 0.04 g/L, more preferably from about 0.015 g/L to about 0.025 g/L. Specific dosages per day can range up to up to 50 mg/day, preferably 1-50 mg/day; more preferably 5-25 mg/day.

Preferably, vitamin B6 can be administered in an amount such that its local concentration in the colon is at least 0.003 g/l, preferably at least 0.007 g/l, most preferably at least 0.01 g/L. Preferred local concentrations in the colon range from about 0.003 g/I to about 0.05 g/l, more preferably from about 0.008 g/I to about 0.03 g/l, most preferably from about 0.01 g/I to about 0.02 g/l. Specific dosages per day can range up to 20 mg/day, preferably 0.5-20 mg/day, more preferably 1.7-9 mg/day.

Preferably, vitamin B9 can be administered in an amount such that its local concentration in the colon is at least 0.001 g/l, preferably at least 0.002 g/l, most preferably at least 0.003 g/l. Preferred local concentrations in the colon range from about 0.001 g/I to about 0.01 g/l, more preferably from about 0.003 g/I to about 0.008 g/l, most preferably from about 0.005 g/I to about 0.007 g/l. Specific dosages per day can range up to 5 mg/day, preferably 0.1-5 mg/day, more preferably 0.4-2 mg/day.

Formulations

A suitable formulation may include a high enough dosage so that a portion of the combination of vitamins is absorbed normally, but the remainder is available to the gut microbiome in the intestine at an effective amount. Other formulations include non-oral routes, such as via suppositories or injections. Preferred formulations are delayed release oral formulations.

A used herein, “delayed release” refers to the release of the active agent at a time later than immediately after administration. Preferably, “delayed release” means delivery of the active agent, upon oral administration, to the large intestine, preferably the colon, in a delayed manner relative to an immediate release formulation.

An “enteric layer” is a layer surrounding a core, wherein the core comprises the active agent and the layer confers resistance to gastric juice. An “enteric shell” is a shell or matrix surrounding or encapsulating the active agent, wherein the shell confers resistance to gastric juice. Alternatively, a matrix-based delivery system can be used. Matrix based systems have no discrete layer of coating material but the active agent is more or less homogeneously distributed within the matrix. Further, there are colon-release systems that embed the active agent in e.g. in a fiber matrix (enzyme-triggered) and an enteric coating on top.

In a preferred embodiment for humans, the formulation of the present invention is a solid dosage form for oral administration. The formulation may be in the form of a capsule, pellet, bead, sphere, mini spheres, tablet, mini tablet, or granule, optionally coated with a delayed release coating or shell that prevents the release of the active agent before the small intestine, preferably before the colon.

Coating, shell, or matrix materials for the delayed release of the active agent, in particular for targeted release in the ileum or the large intestine, upon oral administration are known in the art. They can be subdivided into coating materials that disintegrate above a specific pH, coating materials that disintegrate after a specific residence time in the gastrointestinal tract and coating materials that disintegrate due enzymatic triggers specific to the microflora of a specific region of the intestines. Coating or shell materials from different categories are commonly used in combinations. Coating or shell materials of these three different categories for targeting to the large intestine have been reviewed for example in Bansal et al. (Polim. Med. 2014, 44, 2, 109-118). In one embodiment of the present invention the delayed release coating comprises at least one component selected from coating materials that disintegrate pH-dependently, coating materials that disintegrate time-dependently, coating materials that disintegrate due to enzymatic triggers in the intestinal environment (e.g. in the intestinal environment of the ileum and the large intestine), and combinations thereof.

Coating materials that disintegrate pH-dependently include polyvinyl acetate phthalate, cellulose acetate trimellitate, hydroxypropyl methylcellulose phthalate HP-50, HP-55 or HP-55S, cellulose acetate phthalate, shellac, hydroxypropyl methylcellulose acetate succinate (HPMCAS), poly(methacrylic acid, ethyl acrylate) 1:1 (Eudragit© L100-55, Eudragit© L30D-55), poly(methacrylic acid, methyl methacrylate) 1:1 (Eudragit© L-100, Eudragit® L12.5), poly(methacrylic acid, methyl methacrylate) 1:2 (Eudragit® S-100, Eudragit® S12,5, and Eudragit® FS30D).

Coating materials that disintegrate time-dependently include Eudragit® RL, Eudragit®RS, and ethylcellulose.

Coating materials that disintegrate due to enzymatic triggers in the large intestinal environment include chondroitin sulfate, pectin, guar gum, chitosan, inulin, lactulose, raffinose, stachyose, alginate, dextran, xanthan gum, locust bean gum, arabinogalactan, cyclodextrin, pullulan, carrageenan, scleroglucan, chitin, curdulan, levan, amylopectin, starch, amylose, resistant starch, and azo compounds being degraded by azo bonds splitting bacteria.

In one embodiment the formulation comprises an enteric capsule, filled with a composition comprising the active agent. The enteric capsule confers resistance against the acidic environment of the stomach. For example, softgel formulations may deliver the active agent in solution and yet offer advantages of solid dosage forms. Softgel capsules are particularly suited for hydrophobic active agents which do not dissolve readily in water. Vitamin K and omega-3 fatty acids are preferably formulated in softgel capsules.

In another embodiment, the formulation is a tablet comprising (i) a core comprising the active agent, and (ii) a delayed release coating such as an enteric coating. This may be a hard gel capsule.

The release of the active agent(s) may be delayed until the small intestine. In another embodiment, the release of the active agent(s) is delayed until the distal small intestine. In yet another embodiment, the release of the active agent(s) is delayed until the large intestine including the colon.

The following non-limiting Examples are presented to better illustrate the invention

### EXAMPLES

### Example 1

Experimental Setup

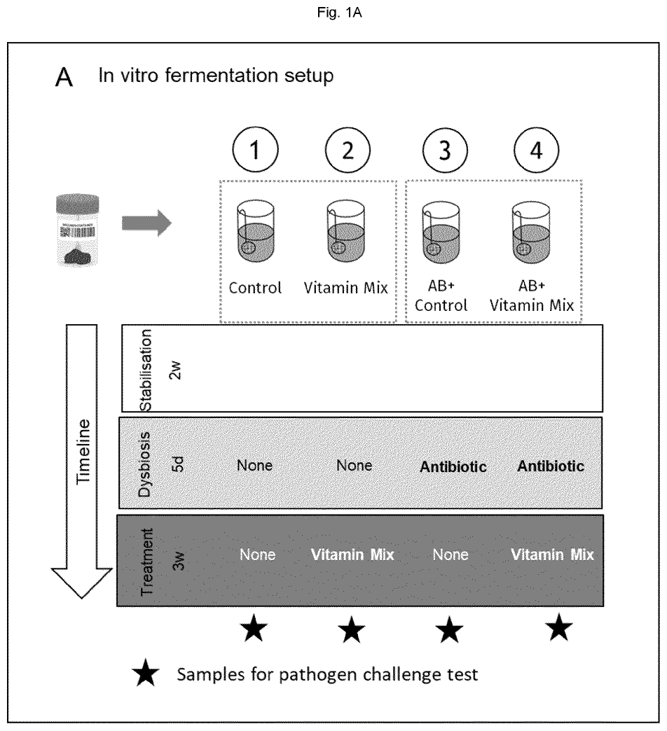

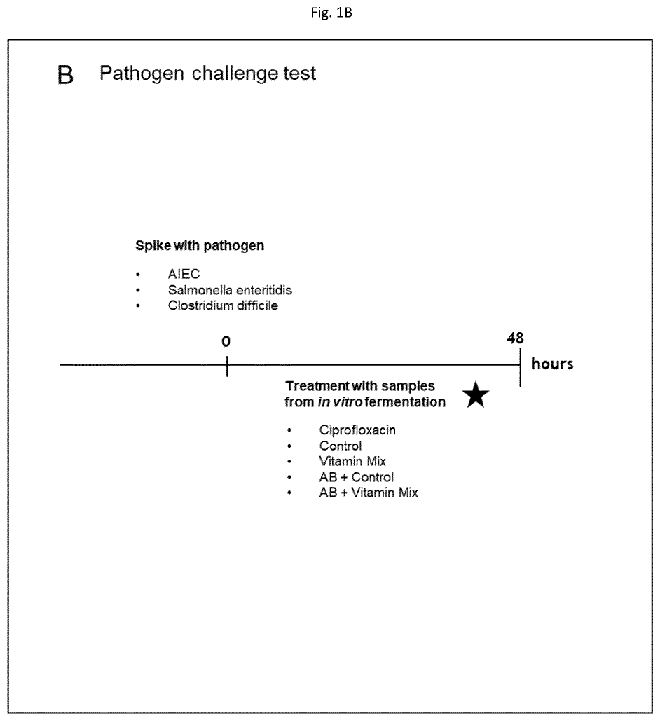

In vitro fermentation study (FIG. 1A)

The properties of a vitamin combination (Vitamin C, Vitamin B2, Vitamin B3, Vitamin B5, Vitamin B6 and Folic Acid) (referred to as “Vitamin Mix”) and a blank control were evaluated in the presence or absence of antibiotic intervention in a fermentation configuration using the microbiota of a healthy adult human donor.

In practice, upon inoculation with a fecal microbiota of a human adult, these reactors simulate the transverse colon (pH 6.15-6.4; retention time=32 h; volume of 800 mL).

The fermentation experiment for this study consisted of three stages:
Stabilization period: After inoculation of the colon reactors with an appropriate fecal sample, a two-week stabilization period allowed the microbial community to differentiate in the different reactors depending on the local environmental conditions. During this period the basic nutritional matrix was provided to the SHIME to support the maximum diversity of the gut microbiota originally present in the faecal inoculum. Analysis of samples at the end of this period allows to determine the baseline microbial community composition and activity in the different reactors.
Antibiotic period: In this four-day period, antibiotic treatment was initiated in arms (3) and (4) of the SHIME and clindamycin was dosed to the colon reactors at a final concentration of 33.9 ppm for a period of 3 days. On day 4, analysis of samples allows to determine the baseline microbial community composition and activity in the different reactors after antibiotic treatment.
Treatment period: During this three-week period, the SHIME reactor was operated under nominal conditions, but with a diet supplemented with the test product. Samples taken from the colon reactors in this period allow to investigate the specific effect on the resident microbial community composition and activity. For the blank control condition, the standard SHIME nutrient matrix was further dosed to the model. Analysis of samples of these reactors allow to determine the nominal microbial community composition and activity in the different reactors, which will be used as a reference for evaluating the treatment effects.

Samples were collected at the end of the last week of treatment for pathogen challenge test.

Treatment with Vitamin Mix includes two weeks of treatment at low dose and one week of treatment at high dose (Table 1).

TABLE 1
Dosage of the different compounds of the test product in the in vitro experiment.

| | Products | Low dose | | | | High dose | | | |
|---|---|---|---|---|---|---|---|---|---|
| Vitamin | Riboflavin | 10 | mg/d | 0.02 | g/L | 50 | mg/d | 0.08 | g/L |
| Mix | (Vitamin B2) | | | | | | | | |
| | Ascorbic Acid | 200 | mg/d | 0.33 | g/L | 1000 | mg/d | 1.67 | g/L |
| | (Vitamin C) | | | | | | | | |
| | Niacinamide | 5 | mg/d | 0.01 | g/L | 25 | mg/d | 0.04 | g/L |
| | (Vitamin B3) | | | | | | | | |
| | Pantothenic acid | 5 | mg/d | 0.01 | g/L | 25 | mg/d | 0.04 | g/L |
| | (Vitamin B5) | | | | | | | | |
| | Pyridoxine | 1.7 | mg/d | 0.003 | g/L | 9 | mg/d | 0.01 | g/L |
| | (Vitamin B6) | | | | | | | | |
| | Folic acid | 0.4 | mg/d | 0.001 | g/L | 2 | mg/d | 0.003 | g/L |

Short-term pathogen challenge test (FIG. 1B)

At the start of the short-term pathogen challenge test, sugar-depleted SHIME nutritional medium containing basal nutrients that are present in the colon (e.g. host-derived glycans such as mucin) was inoculated with a 2% inoculum of an overnight grown pathogenic culture (adherent-invasive E. coli (AIEC) in NB broth, Salmonella enteritidis in BHI broth and Clostridium difficile in RCM broth). Depending on the test condition, a SHIME-derived microbiota or a positive control (antibiotic ciprofloxacin was added at a concentration of 125 ppm) were added. The specific effect of the different test conditions was assessed upon a simulated colonic incubation of 48 h, at 37° C., under shaking and anaerobic conditions. The incubations were performed in fully independent reactors with sufficiently high volume (50 mL) in order to not only allow for a robust microbial fermentation, but also to allow for collecting multiple samples over time.

Pathogenic colonization was assessed through spread plating. Samples from the colonic incubations were collected after 0 (pathogenic inoculum) and 48 h to determine the number of colony forming units (CFU) of the pathogenic strains. Ten-fold dilution series were prepared from these samples in anaerobic phosphate buffered saline and subsequently transferred to petri dishes containing selective agar media, i.e. McConkey agar supplemented with ampicillin and erythromycin for AIEC, McConkey agar supplemented with streptomycin for Salmonella enteritidis and BD 254406 C. difficile selective medium for Clostridium difficile. Selective quantification of the bacterial strains was performed through spread plating on the selective medium, plates were subsequently incubated at 37° C. for at least 24 h under aerobic conditions for AIEC and Salmonella enteritidis and under anaerobic conditions for Clostridium difficile.

Results

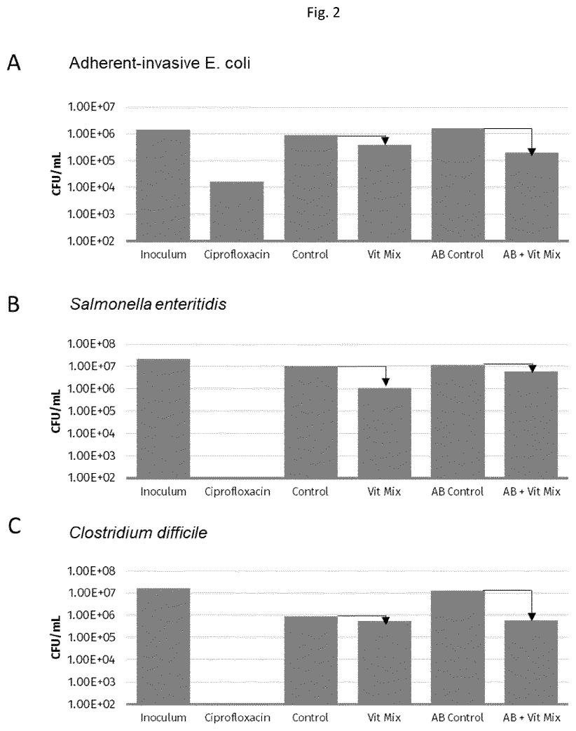

Growth-inhibitory effect of Vitamin Mix on pathogens (FIG. 2)

Adherent-invasive E. coli (AIEC) (FIG. 2A):
As positive control, the broad-spectrum antibiotic ciprofloxacin decreased abundance of AIEC.
Vitamin Mix, when added to the healthy (without exposure to antibiotic) and dysbiosed microbiota (previously treated with antibiotics), exerted inhibitory effects on AIEC growth, with decreases of 0.37 log₁₀ CFU and 0.93 log₁₀ CFU compared to the counterpart control, respectively.

Salmonella enteritidis (FIG. 2B):
As positive control, the broad-spectrum antibiotic ciprofloxacin strongly decreased abundance of Salmonella enteritidis.
Vitamin Mix, when added to the healthy (without exposure to antibiotic) and dysbiosed microbiota (previously treated with antibiotics), exerted inhibitory effects on growth of Salmonella enteritidis, with decreases of 0.96 log₁₀ CFU and 0.28 log₁₀ CFU compared to the counterpart control, respectively.

Clostridium difficile (FIG. 2C):
As positive control, the broad-spectrum antibiotic ciprofloxacin strongly decreased abundance of Clostridium difficile.
Vitamin Mix, when added to the dysbiosed microbiota (previously treated with antibiotics), exerted inhibitory effects on growth of Clostridium difficile, with decreases of 1.3 log₁₀ CFU compared to the counterpart control.

1. A method of decreasing a population of pathogenic bacteria present in the intestine comprising administering a composition comprising Vitamin C directly to the intestine.

2. A method according to claim 1 comprising administering a composition comprising Vitamin C, Vitamin B2 and/or Vitamin B3 directly to the intestine.

3. A method according to claim 1 comprising administering a composition comprising of Vitamin C, Vitamin B2, Vitamin B3, Vitamin B5, Vitamin B6 and folic acid (“Vitamin Mix”) directly to the large intestine.

4. A method according to claim 1 wherein the bacteria which are decreased are selected from the group consisting of: as Adherent Invasive E. coli (“AIEC”), Salmonella enteritidis and/or Clostridium difficile.

5. A method according to claim 4 wherein the pathogen is associated with a disease or adverse condition selected from the group consisting of: inflammatory bowel disease (Crohn's disease and ulcerative colitis), Clostridium difficile-related diarrhea, pseudomembranous colitis, toxic megacolon, perforation of the colon, sepsis, bacterial food poisoning, and bacterial-related traveler's diarrhea and antibiotic associated diarrhea (′AAD″).

6. Use of a directly delivered composition comprising Vitamin C to decrease the population of pathogenic bacteria in the distal gut.

7. Use according to claim 6 where the composition comprises Vitamin C, Vitamin B2 and Vitamin B3.

8. Use according to claim 6 where the composition comprises Vitamin C, Vitamin B2, Vitamin B3, Vitamin B5, Vitamin B6 and folic acid (“Vitamin Mix”)

9. Use of a composition according to claim 6 to prevent or treat diseases or adverse conditions selected from the group consisting of:
inflammatory bowel disease (Crohn's disease and ulcerative colitis), Clostridium difficile-related diarrhea, pseudomembranous colitis, toxic megacolon, perforation of the colon, sepsis, bacterial food poisoning, and bacterial-related traveler's diarrhea and antibiotic associated diarrhea (“AAD”).

10. Use according to claim 6 in a person exposed to antibiotics.
